(19) United States
(12) Patent Application Publication
(10) Pub. No.: US 2023/0304966 A1
(43) Pub. Date: Sep. 28, 2023

(54) DISPLAY METHOD, ANALYZER, AND STORAGE MEDIUM

(71) Applicant: Shimadzu Corporation, Kyoto-shi (JP)

(72) Inventors: Kota Ogino, Kyoto-shi (JP); Akira Harada, Kyoto-shi (JP)

(73) Assignee: Shimadzu Corporation, Kyoto-shi (JP)

(21) Appl. No.: 18/171,811

(22) Filed: Feb. 21, 2023

(30) Foreign Application Priority Data
Mar. 25, 2022 (JP) 2022-049912

(51) Int. Cl. G01N 27/447; G01N 1/40

(52) U.S. Cl. CPC G01N 27/44717; G01N 1/4005; G01N 27/44782; G01N 2001/4038

(57) ABSTRACT

A display method for displaying a plurality of pieces of data respectively obtained by electrophoretic separation for a plurality of samples. The first data includes a first peak corresponding to a separated component of the known sample. The second data includes a second peak corresponding to a separated component of the unknown sample. The display method includes: acquiring the plurality of pieces of data; detecting the first peak and the second peak respectively from the first data and the second data; setting a range included within a predetermined threshold from the first peak as a specified range; determining, for the second data, whether or not the second peak is included in the specified range; and displaying specifically the second peak, when the second peak is in the specified range.

### BACKGROUND OF THE INVENTION

### Field of the Invention

The present invention relates to a display method, an analyzer, and a storage medium.

### Description of the Background Art

Conventionally, software that analyzes and displays electrophoresis data displays detected data in the form of electropherograms, gel images, peak lists, and the like.

Japanese Patent Laying-Open No. 2020-20725 discloses an example of a data analyzer, for a sample separated by electrophoresis, that displays detected data in the form of an electropherogram and a gel image.

### SUMMARY OF THE INVENTION

Such electrophoresis data includes a plurality of peaks corresponding to a plurality of components fractionated by electrophoresis. Usually, a user designates a peak to be focused among the plurality of peaks and analyzes this peak to be focused. Such a method of designating a peak to be focused has been performed, for example, by visually and manually designating a peak corresponding to a component to be focused by a user, but further contrivance is required.

The present disclosure has been made in view of such circumstances, and an object of the present disclosure is to improve work efficiency of a user by displaying automatically and specifically a peak to be focused in an electrophoresis data analyzer.

A first aspect of the present disclosure relates to a display method for displaying a plurality of pieces of data respectively obtained by electrophoretic separation for a plurality of samples. The plurality of samples include a known sample and an unknown sample. The plurality of pieces of data include first data corresponding to the known sample and second data corresponding to the unknown sample. The first data includes a first peak corresponding to a separated component of the known sample. The second data includes a second peak corresponding to a separated component of the unknown sample. The display method includes: acquiring the plurality of pieces of data; detecting the first peak and the second peak respectively from the first data and the second data; setting a range included within a predetermined threshold from the first peak as a specified range; determining, for the second data, whether or not the second peak is included in the specified range; and displaying specifically the second peak, when the second peak is in the specified range.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram illustrating an overall configuration of an analysis system according to the present embodiment.

FIG. 2 is a diagram illustrating an example of a method setting screen.

FIG. 3 is a diagram illustrating an example of a detection setting screen.

FIG. 4 is a diagram illustrating an example of a setting screen of sample information and an analysis order.

FIG. 5 is a diagram illustrating an example of an analysis result screen.

FIG. 6 is a diagram illustrating an example of an electropherogram.

FIG. 7 is a flowchart showing display processing of an analyzer.

FIG. 8 is a flowchart showing a detection range setting process.

FIG. 9 is a diagram illustrating an example of an analysis result screen according to a modified example.

FIG. 10 is a flowchart showing processing related to post-setting of control.

### DESCRIPTION OF THE PREFERRED EMBODIMENTS

Embodiments of the present invention will be described in detail with reference to the drawings. The same or corresponding parts in the drawings are denoted by the same reference numerals, and descriptions thereof will not be repeated.

[1. Configuration of Analysis System]

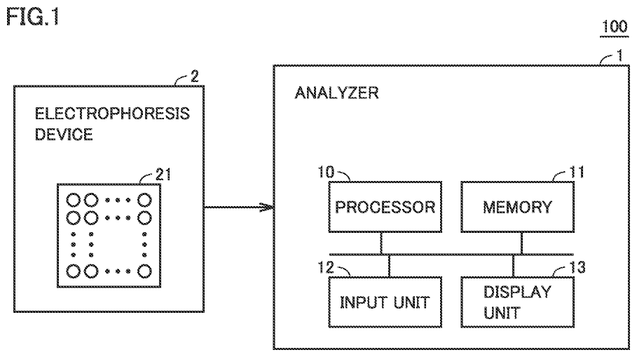

FIG. 1 is a diagram illustrating an overall configuration of an analysis system. As shown in FIG. 1, an analysis system 100 includes an analyzer 1 and an electrophoresis device 2.

Electrophoresis device 2 automatically and continuously analyzes a plurality of samples placed on a plate 21 on which wells are provided in a matrix. Specifically, for example, a user places the samples on plate 21. Electrophoresis device 2 introduces the samples into a fine analysis channel from plate 21. The analysis channel contains a fluorescent dye, and the samples are dyed with the fluorescent dye while moving in the analysis channel. Electrophoresis device 2 applies a voltage to the analysis channel and separates the samples for respective components. Then, electrophoresis device 2 irradiates the analysis channel with ultraviolet light, and detects fluorescence generated from the separated components. Electrophoresis device 2 acquires separation data indicating a relationship between a signal intensity of fluorescence and a moving distance in the analysis channel. The movement distance generally correlates with a size (molecular weight) of the samples.

Here, the samples contain DNA or RNA as a component, and electrophoresis device 2 separates DNA or RNA by a number of base pairs (bp: base pair or nt: nucleotide) that is the size thereof. However, the components of the samples are not limited to the above examples as long as they can be separated by electrophoresis, and may be proteins, for example.

Analyzer 1 acquires the separation data obtained by electrophoresis from electrophoresis device 2 and performs data analysis processing. The separation data corresponds to an example of “data” in the present disclosure.

Analyzer 1 includes a processor 10, a memory 11, an input unit 12, and a display unit 13.

Processor 10 includes, for example, a central processing unit (CPU). Processor extracts programs stored memory 11 into a RAM or the like, and executes the programs.

Memory 11 includes, for example, a read only memory (ROM), a random access memory (RAM), and a nonvolatile memory. The programs stored in the ROM are programs in each of which a processing procedure of analyzer 1 is described. The nonvolatile memory stores the separation data sent from electrophoresis device 2 as a data file. Note that memory 11 may include a hard disk device instead of or in addition to the nonvolatile memory.

Input unit 12 includes, for example, a keyboard, a mouse, and the like. Display unit 13 includes a liquid crystal display or the like. Analyzer 1 generates display data according to a program describing a processing procedure, and causes display unit 13 to display the display data. Control of this procedure is not limited to software processing, and can be processed by dedicated hardware (electronic circuit).

[2. Comparison with Conventional Analysis System]

The separation data obtained by the electrophoretic separation includes peaks corresponding to the separated components. Usually, in the analysis of the separation data, the user visually or manually designates a peak corresponding to a component to be focused among peaks included in the separation data, and analyzes the designated peak.

For example, consider a case of analyzing whether or not the unknown sample contains the same component as that of the known sample which is a control sample (control). In this case, it is determined whether or not the peak included in the separation data of the unknown sample (hereinafter, also referred to as an “unknown sample peak”) and the peak included in the separation data of the control (hereinafter, also referred to as “control peak”) match. In this case, for example, a detection range of the unknown sample peak is set on the basis of the control peak. Then, when the unknown sample peak is included in the detection range, the user detects the unknown sample peak and determines whether or not the unknown sample includes the same component as the control.

Specifically, for example, the user first sets the detection range within a predetermined threshold from the control peak, and inputs the detection range to the analyzer. Then, the analyzer determines whether or not the unknown sample peak is included in the detection range. When the unknown sample peak is included in the detection range, the analyzer detects the unknown sample peak. Therefore, the user can determine whether or not the unknown sample is the same as the control on the basis of the detected peak.

However, in a case where the user manually sets the detection range in this manner, the following problems are conceivable. The first problem is that the user needs to reset the detection range each time the control is changed. The second problem is that when a large number of peaks are included in the separation data of the control, the user needs to set a large number of detection ranges corresponding to this large number of peaks. A third problem is that the user needs to analyze the separation data of the control, detect the control peak, subsequently set a range to be detected based on the control peak, and then again analyze the separation data of the unknown sample. That is, the user has to analyze the sample twice.

Therefore, analyzer 1 according to the present embodiment sets a range included within a predetermined threshold from the control peak automatically as a detection range. Then, in a case where the unknown sample peak is included in the detection range, analyzer 1 displays specifically the unknown sample peak. That is, since analyzer 1 displays automatically and specifically the peak to be focused on, the work efficiency of the user is improved.

[3. Setting Screen]

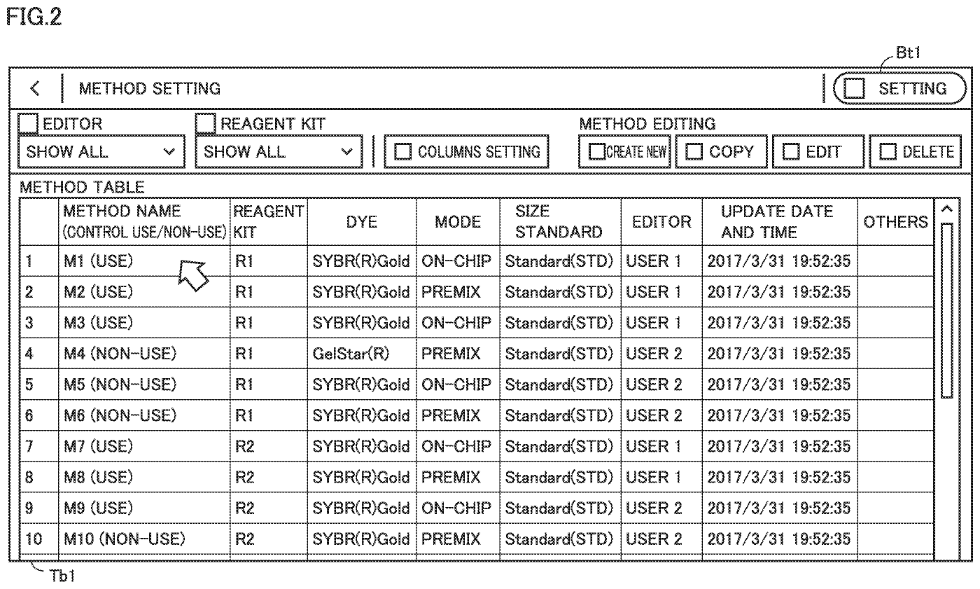

Next, with reference to FIGS. 2 to 4, screens on which the user performs various settings in order to set the detection range from the control peak performed by analyzer 1 and to display specifically the unknown sample peak included in the detection range will be described. Screens illustrated in FIGS. 2 to 4 are displayed on display unit 13 by processor 10. The control peak and the unknown sample peak respectively correspond to examples of a “first peak” and a “second peak” in the present disclosure. Similarly, the separation data of the control and the separation data of the unknown sample respectively correspond to examples of “first data” and “second data” in the present disclosure.

(3-1. Method Setting Screen)

FIG. 2 is a diagram illustrating an example of a method setting screen. The method setting screen is a screen for setting a method used for analysis. The method indicates an analysis method including at least one of a condition related to analysis, information on consumables, and a procedure. The method includes a method table Tb1 and a method setting button Bt1.

Method table Tb1 presents information on the method. Method table Tb1 includes items of “method name (control use/non-use)”, “reagent kit”, “dye”, “mode”, “size standard”, “editor”, “update date and time”, and “others”.

The item “method name (control used/not used)” indicates a name of the method and whether the method is a method using a control or not.

The item “reagent kit” indicates a name of the reagent kit used for the method. The type of reagent kit is determined by a size (bp) of a component to be separated, for example.

The item “dye” indicates a name of a dye used in the method. The dye is used to dye or develop the color of the component to be separated by electrophoretic separation. When the dye is a fluorescent dye, for example, a signal intensity (mV) of fluorescence generated by irradiation with ultraviolet light is detected.

The item “mode” indicates a reagent preparation mode used for the method.

The item “size standard” indicates a name of a size standard used for the method. The size standard is a sample that is used as an index of the size of an unknown sample to be analyzed and a known sample that is used as the control. The size standard as used herein corresponds to an example of a “reference sample” for defining a separated component by the number of base pairs in the present disclosure. The separation data of the size standard corresponds to an example of “third data” in the present disclosure.

The item “editor” indicates a person who has edited the method.

The item “update date and time” indicates date and time at which the method has been updated.

The item “others” indicates other items included in method table Tb1. For example, “others” may include a comment regarding the method.

Method setting button Bt1 is a button used to set a method.

The method setting on the method setting screen is performed, for example, as follows. The user selects a row including a desired method in the method table Tb1 by clicking. Thereafter, the user clicks method setting button Bt1 to perform setting to use the selected method.

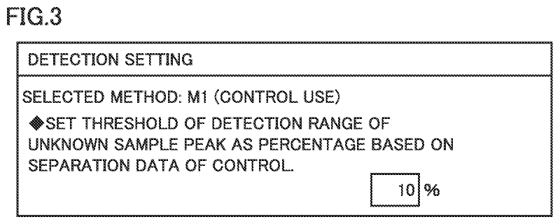

In FIG. 2, when the user selects a method for using the control, a detection setting screen of FIG. 3 is displayed next.

(3-2. Detection Setting Screen)

FIG. 3 is an example of the detection setting screen. The detection setting screen is a screen for performing setting related to the detection range of the unknown sample peak based on the separation data of the control. The setting regarding the detection range includes, for example, setting of a threshold of the detection range.

Specifically, for example, a product obtained by multiplying the size (bp) corresponding to the control peak by a predetermined percentage is set as the threshold. In this case, a range included within the threshold from the control peak is the detection range.

For example, when the user inputs “10%” as the predetermined percentage, if the control peak is 100 bp, the threshold is 10 bp, and the detection range is set to 90 to 110 bp. On the other hand, when the control peak is 200 bp, the threshold is 20 bp, and the detection range is 180 to 220 bp.

The setting of the threshold of the detection range is not limited to the above example, and the size (bp) itself may be directly input. However, an error of the size of the peak in the separation data is considered to be proportional to the size of the peak.

Therefore, as described above, by setting the threshold as a percentage of the size, a more appropriate detection range can be set.

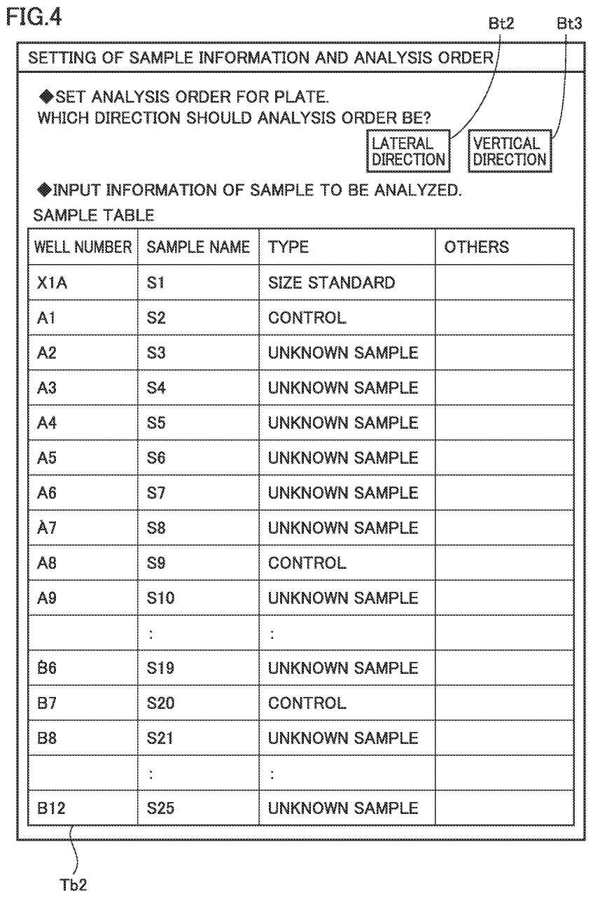

When the user completes the detection setting on the detection setting screen of FIG. 3, or when the user selects the method using the control on the method setting screen of FIG. 2, a setting screen illustrated in FIG. 4 is displayed.

(3-3. Setting of Sample Information and Analysis Order)

FIG. 4 is a diagram illustrating an example of a setting screen of sample information and an analysis order. The setting screen of the sample information and the analysis order includes order setting buttons Bt2 and Bt3 and a sample table Tb2.

Order setting buttons Bt2 and Bt3 are buttons used to set the analysis order. Order setting button Bt2 is configured to set the analysis order of the samples in a lateral direction (row direction) with respect to the plate when clicked. Order setting button Bt3 is configured to set the analysis order of the samples in a vertical direction (column direction) with respect to the plate when clicked.

Sample table Tb2 is a table for the user to input the sample information. Sample table Tb2 includes items “well number”, “sample name”, “type”, and “others”. The names of these items are displayed in cells in a top row of sample table Tb2. Each of the cells in the second and subsequent rows of sample table Tb2 is configured such that the user can input a value corresponding to each item using input unit 12. Each of the cells in the second and subsequent rows of sample table Tb2 may be configured such that the user can select a value corresponding to each item from choices displayed in the cell.

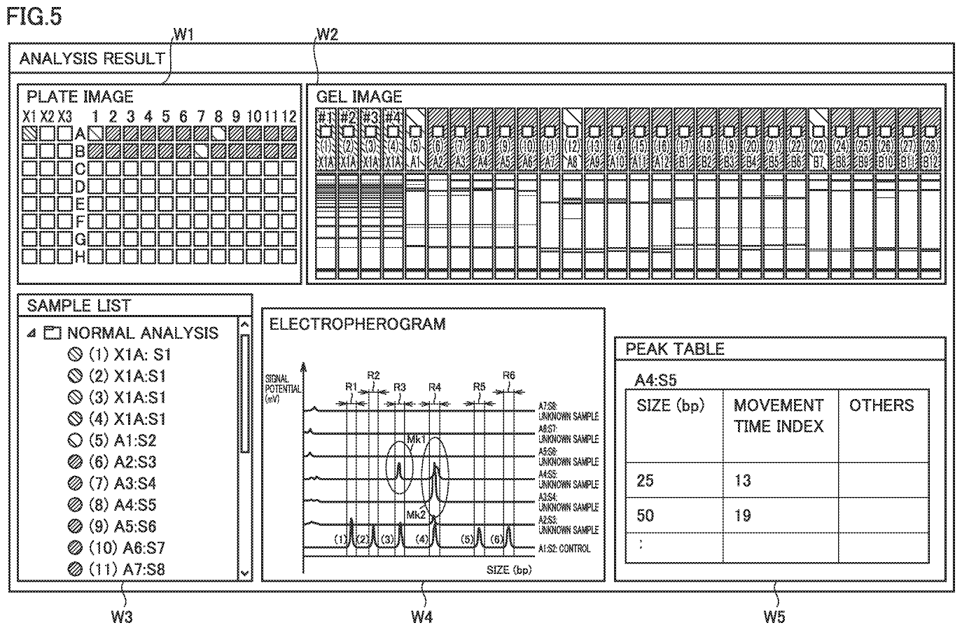

The item “well number” indicates a well number on plate 21 (see the plate image in FIG. 5).

The item “sample name” indicates a name of a sample. An example of the sample name is an ID of the sample allocated to the sample at the time of collecting the sample. Another example of the sample name is the name appropriately named so that the user can easily recognize and manage the sample.

The item “type” indicates a type of the sample. The type of the sample is, for example, a size standard, a control, and an unknown sample.

The item “others” indicates other items included in sample table Tb2. For example, “others” may be comments on the samples.

The setting of the sample information and the analysis order on the setting screen of the sample information and the analysis order is performed as follows, for example. The user clicks order setting button Bt2 or Bt3 to set the analysis order of the samples. Next, the user inputs sample information in sample table Tb2.

The sample information is input as follows, for example. First, the user inputs well numbers to be used for analysis, in the lateral direction with respect to the plate, in view of the analysis order. With this configuration, in electrophoresis device 2, samples are analyzed in order from the top of sample table Tb2. Next, the user determines samples to be placed respectively in the wells. At this time, for each unknown sample, a control for setting a detection range of the unknown sample peak is specified.

Note that specifying a control for setting a detection range of a sample peak in this manner is hereinafter also referred to as “associating an unknown sample with a control”.

In the present embodiment, it is assumed that the unknown sample is associated with a control that has been analyzed recently. That is, in a case where there are one or more unknown samples to be compared with a predetermined control, the one or more unknown samples are input to sample table Tb2 so as to be analyzed immediately after the control. That is, the information of the one or more unknown samples is input to sample table Tb2 following the information of the control. In other words, the information of the one or more unknown samples is input to one or more rows immediately below the row in which the information of the control is input.

In the example of FIG. 4, samples S3 to S8, which are unknown samples, are associated with a sample S2, which is a control. In addition, samples S10 to S19, which are unknown samples, are associated with a sample S9, which is a control. Further, samples S21 to S25, which are unknown samples, are associated with a sample S21, which is a control.

With this configuration, the user can automatically associate the unknown sample with the control only by inputting the sample information in view of the analysis order. In addition, as illustrated in FIG. 5, also in analysis results of the samples arranged in an analysis order, it is easy to compare the control with the unknown sample.

However, the association between the unknown sample and the control is not limited to this example, and for example, the user may set a sample to be used as a control for each unknown sample.

Note that, all of the various settings illustrated in FIGS. 2 to 4 may not be reset for each analysis, and the previously used setting may be automatically used as long as there is no instruction to change the setting. With this configuration, efficiency of analysis under the same condition (routine work) can be improved.

[4. Analysis Result Screen]

(4-1. Analysis Result Screen)

Next, FIG. 5, which is a screen showing a result of analyzing the samples and the separation data by analysis system 100 based on the various settings set as described above, will be described.

FIG. 5 is a diagram illustrating an example of an analysis result screen. The analysis result screen includes windows W1 to W5. Window W1 displays a plate image. Window W2 displays a gel image. Window W3 displays a sample list. Window W4 displays the electropherogram. Window W5 displays a peak table.

The plate image is an image corresponding to an arrangement of the wells in a matrix formed in plate 21 of FIG. 1. The plate image is created based on the setting of the sample information and the analysis order of FIG. 4. In the plate image, the wells in which the samples are placed are shown to have different visual representations depending on the types of the placed samples (size standard, control, and unknown sample). An example of the different visual representations is, for example, a different hatching. Another example of the different visual representations is, for example, a different color. Such different visual representations depending on the types of the samples are also commonly used in gel images and sample lists. Therefore, the user can easily recognize the types of the respective samples in the plate image, the gel image, and the sample list. In addition, it is possible to easily recognize where the sample that is focused in one of the plate image, the gel image, and the sample list is located in the remaining two images. That is, the user can easily recognize the correspondence between the samples in the plate image, the gel image, and the sample list.

In the present embodiment, plate 21 includes wells of rows A to H and columns X1 to X3 and 1 to 12. In the example of FIG. 5, the size standard is installed in a well X1A at row A and column X1. The control and the unknown samples are placed in wells of rows A to B×12 columns.

The sample list is a list in which samples indicated by plate images are arranged in the order of analysis. The sample list includes, for each sample, information of the sample type indicated by hatching, an analysis order indicated in (), a well number, and a sample name.

In the present embodiment, electrophoresis device 2 having four analysis channels and capable of performing electrophoresis of four samples at a time is used. In the example of FIG. 5, first, analysis is performed using four analysis channels for sample S1 that is the size standard, and thus the same well X1A is shown in the analysis orders (1) to (4). Thereafter, sample S2 and subsequent samples are sequentially analyzed once, in the analysis order (5) and subsequent steps.

The gel image represents the separation data of the sample in a simple representation. In the gel image, the analysis order is shown from (1) to (28) and positions of the wells are shown under the analysis order. The gel image is created based on the separation data obtained by electrophoresis device 2. A vertical axis of the gel image is a unit that correlates with migration time, size, or travel distance. The gray level in the gel image correlates with signal strength (mV). Therefore, each of the stripes in the gel image indicates a component contained in the sample.

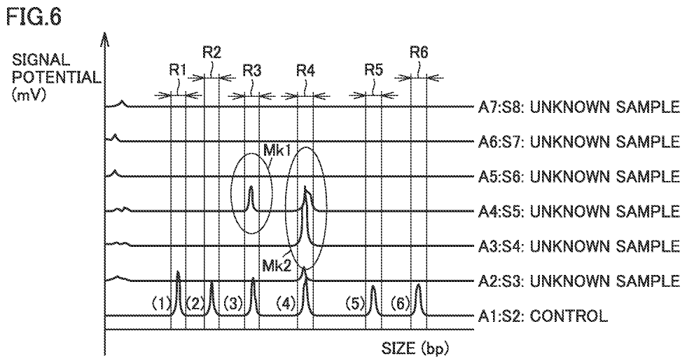

The electropherogram shown in the enlarged view of FIG. 5 or FIG. 6 is an electropherogram of the control and the unknown sample. The electropherogram is created based on the separation data obtained by electrophoresis device 2. A horizontal axis of the electropherogram is a unit correlated with migration time, size, or distance traveled. In the present embodiment, the horizontal axis of the electropherogram is the size (bp). A vertical axis of the electropherogram is the signal intensity (mV).

The size of the component that is the horizontal axis of the electropherogram is calculated, for example, as follows on the basis of the size standard. The size standard includes a component indicating a peak corresponding to a plurality of predetermined sizes (bp) in the separation data. Therefore, the correlation between the size (bp) of the component and the migration time in the analysis channel can be calculated from the size standard separation data. Based on this correlation, the migration times included in the separation data of the control and the unknown sample can be converted to bp.

In the electropherogram, a peak corresponding to the component contained in the sample is shown. For example, the peak is detected, by analyzer 1, using, as an index, a fact that a change in signal intensity per unit time is equal to or greater than a predetermined threshold. The peaks shown in the electropherograms correspond to the stripes in the gel image.

In the present embodiment, in the electropherogram in a state in which the control is set, when the unknown sample peak is included in the detection range included within a predetermined threshold from the control peak, the unknown sample peak is displayed so as to have a different visual representation as compared with the case where the unknown sample peak is not included.

In the examples of FIGS. 5 and 6, electropherograms of sample S2, which is a control placed in a well A1 at row A and column 1, and samples S3 to S8, which are unknown samples placed in wells A2 to A7 at row A and columns 2 to 7, are displayed. In the electropherogram of sample S2, six control peaks marked with symbols (1) to (6) are detected. For control peaks (1) to (6), detection ranges R1 to R6 included within a predetermined threshold from control peaks (1) to (6) are shown. Detection ranges R1 to R6 are set based on the setting on the detection setting screen of FIG. 3, and the unknown sample peaks included in detection ranges R1 to R6 among the unknown sample peaks included in samples S3 to S8 are displayed with markers Mk1 and Mk2 attached thereto. Markers Mk1 and Mk2 indicate that the unknown sample peaks to which markers Mk1 and Mk2 are attached are included in detection ranges R1 to R6. That is, the peak to which the user should pay attention is displayed automatically and specifically. Therefore, the user can easily recognize the peak to be focused on. Therefore, work efficiency of the user is improved.

Note that the different visual representations in the electropherogram are not limited to this example, and for example, the electropherogram may be configured to be drawn in different colors only at a portion of the unknown sample peak included in the detection range. In addition, symbols (1) to (6) and detection ranges R1 to R6 are displayed in FIGS. 5 and 6 for the sake of explanation, but may not be displayed on display unit 13 of actual analyzer 1.

Referring again to FIG. 5, the peak table is a table including values corresponding to peaks. The peak table includes, for example, items “size (bp)”, “movement time index”, and “others”. The item “size (bp)” indicates a size (bp) of the peak included in the detection region. The item “movement time index” is an index of migration time in the analysis channel, and is used to calculate the size (bp). The item “others” indicates other items included in the peak table. For example, the item “others” may include a concentration value of a component corresponding to a peak calculated from the signal intensity of the peak included in the detection range.

In the present embodiment, in a state where the control is set, the peak table is configured to display the corresponding value only when the unknown sample peak is included in the detection range. In the example of FIG. 5, the peak table of S5 arranged in well A4 at row A and column 4 is illustrated. With this configuration, the user can easily recognize the peak to be focused on also in the peak table. Therefore, work efficiency of the user is improved.

[5. Flow of Display Processing of Analyzer]

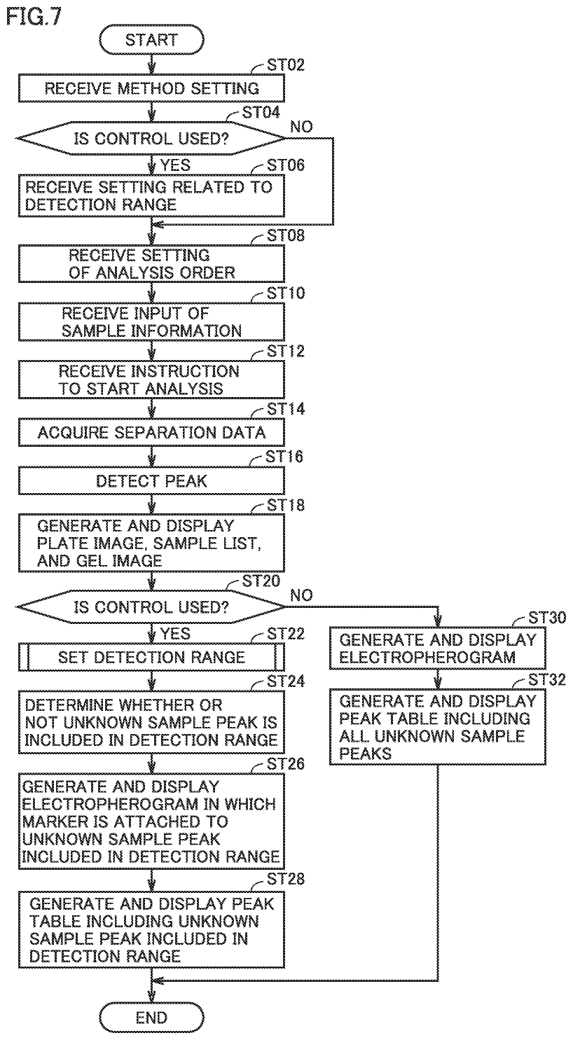

FIG. 7 is a flowchart illustrating display processing of the analyzer. Referring to FIG. 7, in step (hereinafter, also referred to as ST) 02, processor 10 receives method setting by the user by using the method setting screen (FIG. 2).

In ST04, processor 10 determines whether or not the method set in ST02 uses the control.

If the method uses the control (YES in ST04), in ST06, processor 10 receives setting related to the detection range of the peak by the user, using the detection setting screen (FIG. 3). The setting regarding the detection range includes setting of the threshold of the detection range.

When the method does not use the control (NO in ST04), or following ST06, in ST08, processor 10 receives the setting of the analysis order by the user by using the setting screen of the sample information and the analysis order (FIG. 4).

In ST10, processor 10 receives the input of the sample information by the user using the setting screen of the sample information and the analysis order (FIG. 4). The sample information includes the type of the sample.

Before subsequent ST12, the user installs the samples on plate 21 in view of the setting of the sample information and the analysis order. Then, the user installs plate 21 in electrophoresis device 2.

In ST12, processor 10 receives an instruction to start analysis. The instruction to start the analysis is performed, for example, when the user clicks an analysis start button (not illustrated) displayed on display unit 13.

In ST14, processor 10 acquires the separation data obtained by electrophoretic separation from electrophoresis device 2.

In ST16, processor 10 detects peaks included in the separation data of the size standard, the control, and the unknown samples.

In ST18, processor 10 generates a plate image, a sample list, and a gel image (FIG. 5), and displays the plate image, the sample list, and the gel image on display unit 13.

In ST20, processor 10 determines whether or not the method set in ST02 uses the control.

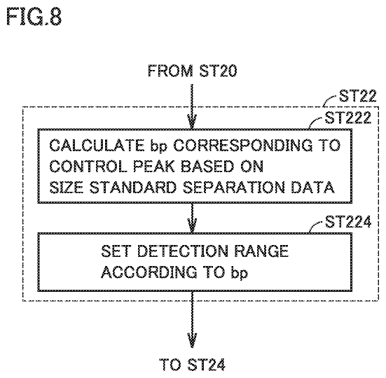

If the method uses a control (YES in ST20), in ST22, processor 10 sets, as the detection range, a range included within the predetermined threshold set in ST06 from the control peak. Detailed processing regarding the setting of the detection range will be described with reference to FIG. 8.

In ST24, processor 10 determines whether or not the unknown sample peak is included in the set detection range.

In ST26, processor 10 generates an electropherogram in which a marker is attached to an unknown sample peak included in the detection range, and displays the electropherogram on display unit 13. In other words, in the electropherogram, when the unknown sample peak is included in the detection range, the unknown sample peak is displayed with a marker.

In ST28, processor 10 generates a peak table including the unknown sample peak included in the detection range, and displays the peak table on display unit 13. In other words, the peak table is configured to include a value corresponding to the unknown sample peak only when the unknown sample peak is included in the detection range. When ST28 is completed, processor 10 ends the processing.

If the method does not use a control (NO in ST20), processor 10 generates an electropherogram and displays the electropherogram on display unit 13 in ST30. In this case, unlike ST26, the electropherogram is displayed without a marker.

In ST32, a peak table including all unknown sample peaks is generated, displayed on display unit 13, and the process ends.

FIG. 8 is a flowchart showing the detection range setting process.

In ST222, processor 10 calculates bp corresponding to the control peak based on the size standard separation data. Specifically, for example, processor 10 first converts the migration time of the separation data obtained by the electrophoretic separation into a corresponding size (bp). Then, processor 10 calculates bp corresponding to the control peak on the basis of the converted separation data.

In ST224, processor 10 sets a detection range according to bp. The threshold set by a predetermined percentage in ST06 is converted to a detection range expressed in bp for each control peak.

As described above, analyzer 1 according to the present embodiment automatically sets the detection range of the unknown sample peak based on the separation data of the control. Then, analyzer 1 displays automatically and specifically the unknown sample peak included in the detection range. As a result, the user can easily recognize whether or not the unknown sample peak having the same size as the control peak is detected. That is, by analyzer 1 detecting automatically and displaying specifically the peak to be focused by the user, the work efficiency of the user is improved.

[6. Modified Example]

In the above embodiment, in analyzer 1, the control for setting the detection range of the unknown sample peak is set before the separation data is acquired by electrophoresis device 2. However, analyzer 1 can be configured to be able to set the control even after the analysis of the separation data. In the present specification, the setting of the control performed after the analysis of the separation data is also referred to as “post-setting” of the control.

In the modified example, processing of analyzer 1 regarding the post-setting of the control will be described.

(6-1. Control Post-setting Screen)

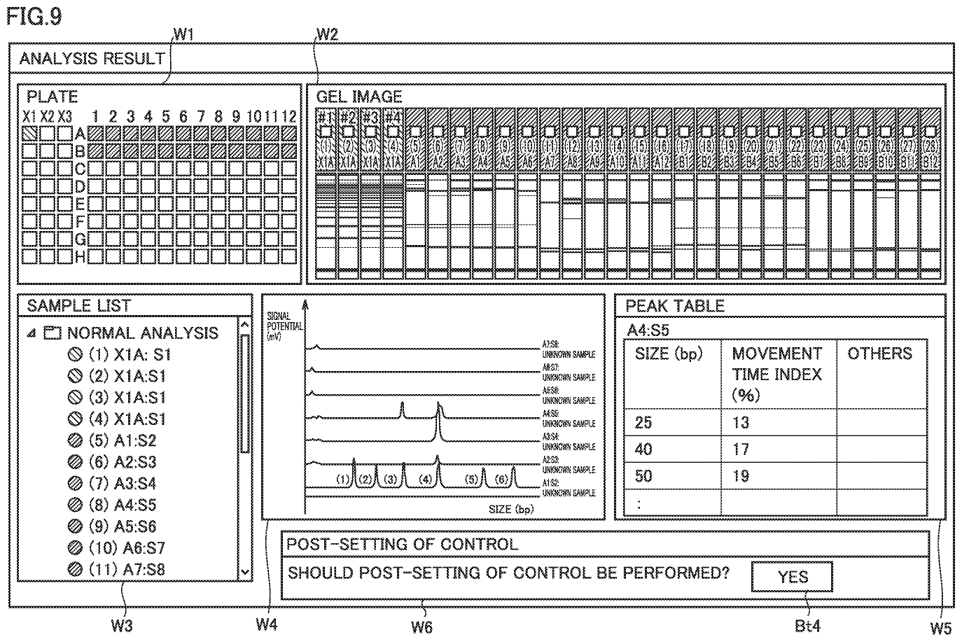

FIG. 9 is a diagram illustrating an example of an analysis result screen according to the modified example. FIG. 9 illustrates a screen showing an analysis result of the same sample as the analysis result screen shown in FIG. 5. However, unlike FIG. 5, FIG. 9 shows a result of analysis performed in a state where the control is not set in advance. This state is, for example, a state in which the samples of wells A2, A8, and B7, which should actually be controls, are erroneously set as unknown samples on the setting screen of the sample information and the analysis order (FIG. 4).

In this case, in the plate image, the gel image, and the sample list, the same hatching as that of the unknown sample is applied to the samples of wells A2, A8, and B7. The electropherogram does not include detection ranges R1 to R6 and markers Mk1 and Mk2. In addition, all peaks of unknown sample S5 are displayed in the peak table.

Further, unlike FIG. 5, FIG. 9 includes a window W6. Window W6 is a window for performing the post-setting of the control. Window W6 includes a post-setting button Bt4 for performing post-setting.

The post-setting in window W6 is performed, for example, as follows. The user refers to other windows W1 to W5 of the analysis result screen, to determine whether or not to perform the post-setting of the control. When the user determines to perform the post-setting, the user can perform the post-setting using post-setting button Bt4. Specifically, for example, when post-setting button Bt4 is clicked by the user, processor displays the setting screen of the sample information and the analysis order (FIG. 4). The user can change the sample information using sample table Tb2 included in the screen. For example, the user changes the sample type from the unknown sample to the control for the samples in wells A2, A8, and B7 that should be controls. In this state, when the user designates to reflect this change in the analysis result screen, the display contents of windows W1 to W5 of the analysis result screen are changed and displayed. That is, the analysis result screen, in which the samples of the wells A2, A8, and B7 illustrated in FIG. 5 are correctly set as the control, is displayed on display unit 13.

Note that the state in which the user does not erroneously set the control is not limited to the above example, and may be, for example, a state in which a method that does not use the control is erroneously set. In this case, when post-setting button Bt4 is clicked, for example, the method setting screen is displayed, and the method setting can be changed. When the user designates to reflect the change in the analysis result screen, the analysis result screen in which the control is correctly set is displayed on display unit 13.

(6-2. Flow of Processing for Post-setting of Control)

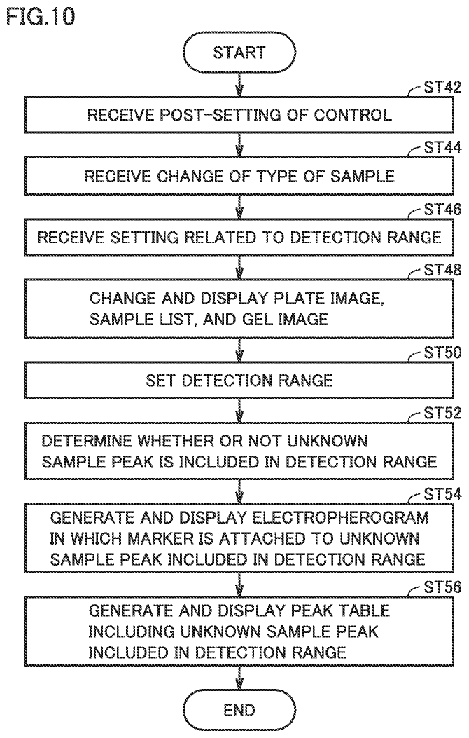

FIG. 10 is a flowchart illustrating processing related to the post-setting of the control. Referring to FIG. 10, in ST42, processor 10 receives the post-setting of the control by the user using post-setting button Bt4 (FIG. 9).

In ST44, processor 10 receives a change of the type of the sample by the user using sample table Tb2 (FIG. 3).

In ST46, processor 10 displays the detection setting screen (FIG. 2) on display unit 13, and receives the setting related to the detection range by the user. The setting regarding the detection range includes, for example, setting of the threshold of the detection range. However, as the setting regarding the detection range, the previously used setting may be used as it is.

In ST48, processor 10 changes and displays the plate image, the sample list, and the gel image on display unit 13.

In ST50, processor 10 sets a range included within the predetermined threshold set in ST46 from the control peak as a detection range.

In ST52, processor 10 determines whether or not the unknown sample peak is included in the detection range.

In ST54, processor 10 generates an electropherogram in which a marker is attached to an unknown sample peak included in the detection range, and displays the electropherogram on display unit 13. Specifically, for example, processor 10 performs a change of adding the marker to the electropherogram already generated in the first analysis.

In ST56, processor 10 generates a peak table including the unknown sample peaks included in the detection range, and displays the peak table on display unit 13. Specifically, for example, processor 10 performs a change to remove a peak that is not included in the detection range from the peak table already generated in the first analysis. When ST56 is completed, processor 10 ends the processing.

As described above, in such a case where the setting of the control before the electrophoretic separation is forgotten, analyzer 1 according to the modified example can set the control after the separation data is analyzed. Therefore, even after the analysis of the separation data, analyzer 1 can display automatically and specifically the peak to be focused by the user, and the work efficiency of the user is improved.

[Aspects]

It is understood by those skilled in the art that the plurality of exemplary embodiments described above are specific examples of the following aspects.

(Clause 1) A display method according to one aspect is a display method for displaying a plurality of pieces of data respectively obtained by electrophoretic separation for a plurality of samples. The plurality of samples include a known sample and an unknown sample. The plurality of pieces of data include first data corresponding to the known sample and second data corresponding to the unknown sample. The first data includes a first peak corresponding to a separated component of the known sample. The second data includes a second peak corresponding to a separated component of the unknown sample. The display method includes: acquiring the plurality of pieces of data; detecting the first peak and the second peak respectively from the first data and the second data; setting a range included within a predetermined threshold from the first peak as a specified range; determining, for the second data, whether or not the second peak is included in the specified range; and displaying specifically the second peak, when the second peak is in the specified range

According to the display method in Clause 1, the second peak is specifically displayed, when a peak of the second data (second peak) is included in the specified range included within the predetermined threshold from the peak detected in the first data (first peak). That is, the peak to be focused by the user is displayed automatically and specifically. Therefore, work efficiency of the user is improved.

(Clause 2) In the display method according to Clause 1, the known sample and the unknown sample each include DNA or RNA. The plurality of samples include a reference sample for defining a separated component by a number of base pairs. The plurality of pieces of data include third data corresponding to the reference sample. The setting includes: calculating a number of base pairs corresponding to the first peak in the first data, based on the third data; and setting the specified range according to the number of base pairs.

According to the display method in Clause 2, in the unknown sample including DNA or RNA, the specified range according to the number of base pairs is set, and the second peak included in the specified range is specifically displayed, and thus the work efficiency of the user can be improved.

(Clause 3) In the display method according to Clause 1 or 2, the displaying specifically further includes displaying an electropherogram based on the second data. The displaying an electropherogram includes displaying a different visual representation, when the second peak is included in the specified range, as compared with a case where the second peak is out of the specified range.

According to the display method in Clause 3, the user can easily focus on the second peak, which is included in the specified range and which is a peak to be focused, by a different visual representation. Therefore, the work efficiency of the user is improved.

(Clause 4) In the display method according to Clause 3, the displaying a different visual representation includes displaying the second peak included in the specified range with a marker in the electropherogram.

According to the display method in Clause 4, the user can easily focus on the second peak, which is included in the specified range and which is a peak to be focused, by the marker. Therefore, the work efficiency of the user is improved.

(Clause 5) In the display method according to Clause 3 or 4, the displaying specifically further includes displaying a peak table based on the second data. The peak table includes a value corresponding to the second peak, only when the second peak is included in the specified range.

According to the display method in Clause 5, the user can refer to only the second peak included in the specified range which is a peak to be focused, in the peak table. Therefore, the work efficiency of the user is improved.

(Clause 6) An analyzer according to another aspect is an analyzer that analyzes a plurality of pieces of data respectively obtained by electrophoretic separation for a plurality of samples. The analyzer includes a memory, a processor, and a display unit. The memory stores the plurality of pieces of data. The processor executes the display method according to any one of Clauses 1 to 5. The display unit displays specifically the second peak included in the specified range.

(Clause 7) A storage medium according to still another aspect records a program executed by a computer to cause the computer to perform the display method according to any one of Clauses 1 to 5.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A display method for displaying a plurality of pieces of data respectively obtained by electrophoretic separation for a plurality of samples,
wherein the plurality of samples include a known sample and an unknown sample,
the plurality of pieces of data include first data corresponding to the known sample and second data corresponding to the unknown sample,
the first data includes a first peak corresponding to a separated component of the known sample, and
the second data includes a second peak corresponding to a separated component of the unknown sample, and
the display method comprises:
acquiring the plurality of pieces of data;
detecting the first peak and the second peak respectively from the first data and the second data;
setting a range included within a predetermined threshold from the first peak as a specified range;
determining, for the second data, whether or not the second peak is included in the specified range; and
displaying specifically the second peak, when the second peak is in the specified range.

2. The display method according to claim 1, wherein
the known sample and the unknown sample each include DNA or RNA,
the plurality of samples include a reference sample for defining a separated component by a number of base pairs,
the plurality of pieces of data include third data corresponding to the reference sample, and
the setting includes:
calculating a number of base pairs corresponding to the first peak in the first data, based on the third data; and
setting the specified range according to the number of base pairs.

3. The display method according to claim 1, wherein
the displaying specifically further includes displaying an electropherogram based on the second data, and
the displaying an electropherogram includes displaying a different visual representation, when the second peak is included in the specified range, as compared with a case where the second peak is out of the specified range.

4. The display method according to claim 3, wherein the displaying a different visual representation includes displaying the second peak included in the specified range with a marker in the electropherogram.

5. The display method according to claim 3, wherein
the displaying specifically further includes displaying a peak table based on the second data, and
the peak table includes a value corresponding to the second peak, only when the second peak is included in the specified range.

6. An analyzer that analyzes a plurality of pieces of data respectively obtained by electrophoretic separation for a plurality of samples, the analyzer comprising:
a memory that stores the plurality of pieces of data;
a processor that executes the display method according to claim 1; and
a display unit that displays specifically the second peak included in the specified range.

7. A computer-readable storage medium that records a program executed by a computer to cause the computer to perform the display method according to claim 1.
